(19) United States
(12) Patent Application Publication
(10) Pub. No.: US 2025/0195279 A1
(43) Pub. Date: Jun. 19, 2025

(54) ADHESIVE BASE BANDAGE FOR APPLYING TOP BANDAGES

(71) Applicant: Jemal Abajebel Tahiro, Saint Paul, MN (US)

(72) Inventor: Jemal Abajebel Tahiro, Saint Paul, MN (US)

(21) Appl. No.: 18/543,387

(22) Filed: Dec. 18, 2023

Related U.S. Application Data
(60) Provisional application No. 63/417,381, filed on Oct. 19, 2022.

(51) Int. Cl. A61F 13/0246; A61F 13/00

(52) U.S. Cl. CPC A61F 13/025; A61F 2013/00165

(57) ABSTRACT

A wound dressing has a base bandage and a top bandage. The base bandage may include a top surface and a bottom surface. The base bandage may be configured to adhere to a user's skin via the bottom surface. The top bandage may also include a top surface and a bottom surface. The bottom surface of the top bandage is configured to adhere to the top surface of the base bandage.

### CROSS-REFERENCE TO RELATED APPLICATION

This application claims the benefit of priority of U.S. provisional application No. 63/417,381, filed Oct. 19, 2022, the contents of which are herein incorporated by reference.

### BACKGROUND OF THE INVENTION

### Field of the Invention

The present invention relates to an adhesive base bandage for applying top bandages.

### Description of the Related Art

Pressure ulcers are injuries to skin that result from prolonged pressure on the skin of a user. Treating such ulcers requires meticulous attention. Frequent cleaning and dressing changes may have to be performed for proper healing. Changing the dressing may bring discomfort to the user, especially in sensitive areas where adhesive can cause pain and hair entanglement. Changing the dressing can even cause the skin to get removed with the adhesive.

Thus, there exists a need for a device that facilitates easy replacement of the dressing, without causing discomfort to the user.

### SUMMARY OF THE INVENTION

The following presents a simplified summary of the present disclosure in a simplified form as a prelude to the more detailed description that is presented herein.

In accordance with embodiments of the invention, there is provided a wound dressing having a base bandage and a top bandage. The base bandage may include a top surface and a bottom surface. The base bandage may be configured to adhere to user's skin via the bottom surface. The top bandage may also include a top surface and a bottom surface. The bottom surface of the top bandage may be configured to adhere to the top surface of the base bandage.

The base bandage may further include an opening to expose a wound, and the top bandage may be configured to cover the wound. In some aspects, the opening may be located at a center portion of the base bandage.

In some aspects, the bottom surface of the base bandage may include a first adhesive configured to stick/adhere the base bandage to the user's skin. In addition, the bottom surface of the top bandage may include a second adhesive configured to stick/adhere the top bandage to the top surface of the base bandage.

In some aspects, the base bandage may include one or more tabs disposed at corners of the base bandage. In further aspects, the base bandage may be greater in size relative to the size of the top bandage.

The present disclosure is further directed towards a method to use the wound dressing. The method may include applying a base bandage on a user's skin. The base bandage may include a top surface and a bottom surface. The base bandage may be configured to adhere to the user's skin via the bottom surface. The method may further include applying a first top bandage on the base bandage. The first top bandage may include a top surface and a bottom surface. The bottom surface of the first top bandage may be configured to adhere to the top surface of the base bandage.

In some aspects, the method may further include removing the first top bandage by detaching the first top bandage from the base bandage, and applying a second top bandage on the base bandage.

The present disclosure discloses a wound dressing that facilitates replacement of the dressing without causing discomfort to the user. The wound dressing may include a layered arrangement of bandages (including a base bandage and a top bandage) which facilitates easy replacement of the top bandage without removal of the base bandage. The base bandage may be removed when the wound is properly healed.

In some embodiments, the base bandage may have an image printed thereon and the top bandage may have a second, complementary image printed thereon, such that placement of the top bandage on the base bandage produces a completed image. For example, the base bandage may illustrate a lion's mane and ears, while the top bandage may illustrate a lion's face. Together, the top bandage on the base bandage illustrates the lion's head. In another example, the base bandage may illustrate a turtle's head, legs, and tail, while the top bandage may illustrate a turtle's shell. Together, the top bandage on the base bandage illustrates the turtle from an overhead view.

These and other features, aspects, and advantages of the present invention will become better understood with reference to the following description and appended claims.

### BRIEF DESCRIPTION OF THE DRAWINGS

Illustrative embodiments of the present invention are described herein with reference to the accompanying drawings, in which:

FIG. 1 depicts a top perspective view of an adhesive base bandage in accordance with the present disclosure.

FIG. 2 depicts a bottom perspective view of the adhesive base bandage of FIG. 1 in accordance with the present disclosure.

FIG. 3 depicts a perspective view of positioning of the adhesive base bandage on user's forearm in accordance with the present disclosure.

FIG. 4 depicts a perspective view of positioning of a top bandage on the adhesive base bandage disposed on the user's forearm in accordance with the present disclosure.

FIG. 5 depicts a perspective view of the top bandage placed on the adhesive base bandage in accordance with the present disclosure.

FIG. 6 depicts a perspective view of replacement of the top bandage while keeping the adhesive base bandage in place in accordance with the present disclosure.

FIG. 7 depicts a top view of an alternate embodiment of an adhesive base bandage in accordance with the present disclosure.

FIG. 8 depicts a bottom view of the adhesive base bandage of FIG. 7 in accordance with the present disclosure.

FIG. 9 depicts a top perspective view of yet another embodiment of an adhesive base bandage applied on user's forearm in accordance with the present disclosure.

FIG. 10 depicts another top perspective view of the adhesive base bandage of FIG. 9 in accordance with the present disclosure.

### DETAILED DESCRIPTION OF INVENTION

For a further understanding of the nature and function of the embodiments, reference should be made to the following detailed description. Detailed descriptions of the embodiments are provided herein, as well as, the best mode of carrying out and employing the present invention. It will be readily appreciated that the embodiments are well adapted to carry out and obtain the ends and features mentioned as well as those inherent herein. It is to be understood, however, that the present invention may be embodied in various forms. Therefore, persons of ordinary skill in the art will realize that the following disclosure is illustrative only and not in any way limiting, as the specific details disclosed herein provide a basis for the claims and a representative basis for teaching to employ the present invention in virtually any appropriately detailed system, structure or manner. It should be understood that the devices, materials, methods, procedures, and techniques described herein are presently representative of various embodiments. Other embodiments of the disclosure will readily suggest themselves to such skilled persons having the benefit of this disclosure.

Reference will now be made in detail to the present preferred embodiments of the invention, examples of which are illustrated in the accompanying drawings. Wherever possible, the same reference numerals are used in the drawings and the description to refer to the same or like parts.

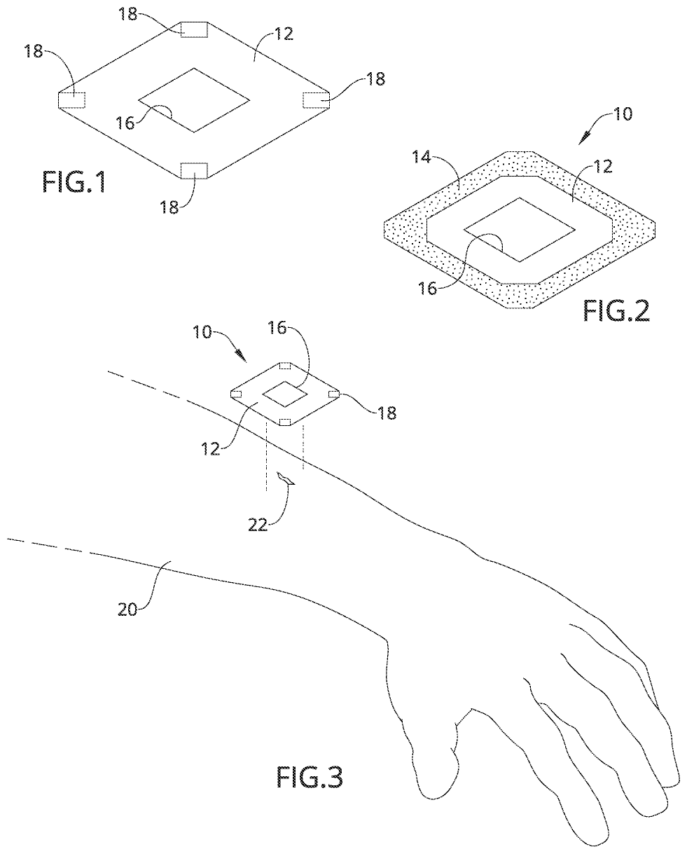

FIG. 1 depicts a top perspective view of an adhesive base bandage 10 (or base bandage 10) in accordance with the present disclosure. FIG. 1 will be explained in conjunction with FIGS. 2-6.

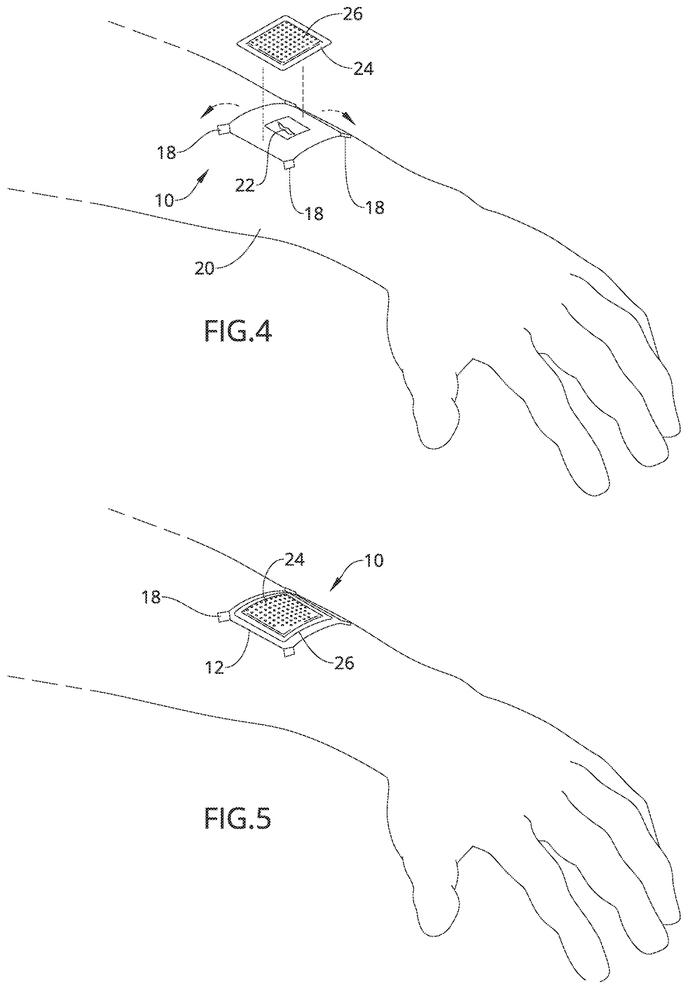

The base bandage 10 may be a bandage that may serve as a surrogate skin on which a top bandage (e.g., a top bandage 24, shown in FIG. 4) may be applied. Stated another way, the base bandage 10 may act as an area for application of the top bandage 24 (or wound dressing). The base bandage 10 may be a bottom stationary layer that may be placed around a treatment area 22 such that the base bandage 10 may surround the treatment area 22 without contacting the treatment area 22 (as shown in FIG. 3). The treatment area 22 may be a wound, a cut, an incision area, etc. The top bandage 24 may be placed over the base bandage 10, and may cover the treatment area 22. Stated another way, the base bandage 10 may be adhered to the skin of a user 20 and the top bandage 24 may be adhered to the base bandage 10 (and not directly to the skin of the user 20). The top bandage 24 may be replaced/changed without removing the base bandage 10.

In some aspects, the base bandage 10 may be made of surrogate skin material 12. In some aspects, the base bandage 10 may be made of material such as silicone foam or non-woven fiber. In addition, the base bandage 10 may be waterproof and alcohol resistant non-allergenic bottom layer. The base bandage 10 may be of any size and shape to fit the various sizes and shapes of the treatment area 22. For example, the base bandage 10 may have a shape of a square, a rectangle, a circle, an oval, a triangle, etc.

In some aspects, the base bandage 10 may include a top surface and a bottom surface (opposite to the top surface). The base bandage 10 may include an adhesive 14 (as shown in FIG. 2) at the bottom surface of the base bandage 10. In some aspects, the adhesive 14 may be disposed on an entire bottom surface of the base bandage 10. Alternatively, the adhesive 14 may be disposed at the edges of the base bandage 10 (e.g., at all the edges of the base bandage 10). The base bandage 10 may be applied to the skin of the user 20 via the adhesive 14.

In some aspects, the base bandage 10 may include an opening 16 to expose the treatment area 22. In some aspects, the opening 16 may be disposed at a center portion of the base bandage 10. Stated another way, the base bandage 10 may surround the treatment area 22 and may not cover (or contact) the treatment area 22. Thus, there may exist a gap between the treatment area 22 and the edges of the base bandage 10. The opening 16 may be of any size and any shape. In some aspects, the shape of the opening 16 and the shape of the base bandage 10 may be same or different.

In some aspects, the base bandage 10 may include one or more tabs 18. In some aspects, the tabs 18 may be disposed at the top surface of the base bandage 10. In some aspects, the tabs 18 may be configured to fold out such that a first portion of the tabs 18 may be at the top surface of the base bandage 10, and a second portion of the tabs 18 may be configured to adhere to the user's skin. The second portion may include adhesive at a bottom surface of the tabs 18 to enable the second portion of the tabs 18 to stick/adhere to the user's skin and facilitate sticking of the base bandage 10 onto the user's skin.

In some aspects, the tabs 18 may be disposed at a predetermined distance from each other. When the base bandage 10 may be square or rectangular in shape, the tabs 18 may be disposed at the corners (or edges) of the base bandage 10. The base bandage 10 may include any count of the tabs 18. For example, the base bandage 10 may include four tabs disposed at the four corners of the base bandage 10. In this case, each tab may be disposed at an equal distance from adjacent tab. When the base bandage 10 may be circular in shape, the tabs 18 may be disposed at the periphery or perimeter of the circular base bandage, as depicted in FIGS. 7 and 8.

FIG. 4 depicts a perspective view of positioning of the top bandage 24 on the adhesive base bandage 10 on the user's forearm in accordance with the present disclosure. The top bandage 24 may be configured to cover the opening 16 (e.g., to cover the treatment area 22). In some aspects, the dimensions of the top bandage 24 may be different from the dimensions of the base bandage 10. The base bandage 10 may be larger in size relative to the size of the top bandage 24. In an exemplary aspect, a length of the base bandage 10 may be 0.75 cm bigger than the length of the top bandage 24. In further aspects, the material of the top bandage 24 and the material of the base bandage 10 may be same or different. In further aspects, the shape of the top bandage 24 may be same as the shape of the base bandage 10, to facilitate secure attachment of the top bandage 24 with the base bandage 10. As an example, the top bandage 24 may be square, rectangular, or circular in shape. In some aspects, the top bandage 24 may not include any opening and may cover the treatment area 22 completely, and may stick to the top surface of the base bandage 10.

The top bandage 24 may include a top surface and a bottom surface. The bottom surface may include an adhesive 26 to adhere the top bandage 24 on the base bandage 10 (e.g., on the top surface of the base bandage 10). The adhesive 26 may be present on a portion of the bottom surface of the top bandage 24. For example, the adhesive 26 may be present on the edges of the top bandage 24. The edges of the top bandage 24 may align with the portion of the edges of the base bandage 10, to enable sticking of the top bandage 24 to the base bandage 10 (e.g., on the top surface of the base bandage 10). In some aspects, the adhesive 26 on the bottom surface of the top bandage 24 may not stick to the user's skin and may stick only to the top surface of the base bandage 10 (e.g., on the edges of the base bandage 10, as shown in FIGS. 4-5) to enable easy replacement of the top bandage 24.

In some aspects, the top bandage 24 may include adsorbent pad that may fit in the opening 16 of the base bandage 10. Stated another way, the dimensions of the adsorbent pad may be equivalent to the dimensions of the opening 16 so that the adsorbent pad may cover the treatment area 22 for proper healing.

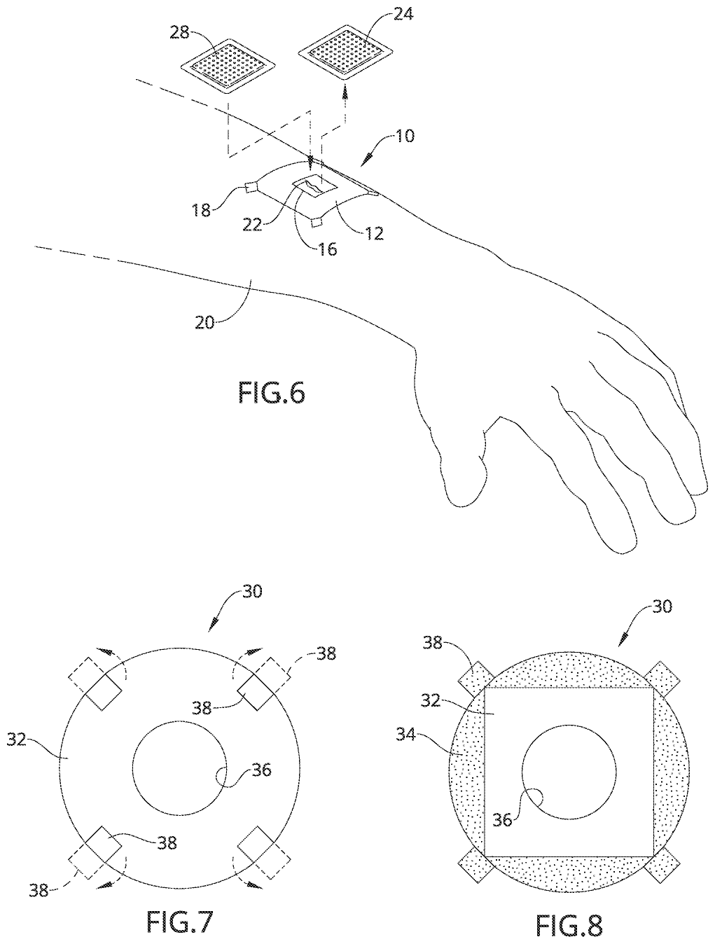

In operation, the user 20 (or any other user) may first apply the base bandage 10 on the user's skin via the adhesive 14, such that the opening 16 may expose the treatment area 22. Stated another way, the edges of the base bandage 10 may surround the treatment area 22 (as depicted in FIG. 3). The base bandage 10 may be applied such that the adhesive 14 may not contact the treatment area 22. When the base bandage 10 may be applied on the user's skin, the user 20 may apply the top bandage 24 over the base bandage 10, via the adhesive 26. Stated another way, the user 20 may adhere the top bandage 24 on the base bandage 10 (as depicted in FIGS. 4-5), and not on the user's skin. When the top bandage 24 is to be replaced, the user 20 may remove the top bandage 24 by detaching the top bandage 24 from the base bandage 10. Once the top bandage 24 is removed, the user 20 may stick another top bandage 28 on the top surface of the base bandage 10 (in a manner similar to sticking the top bandage 24 on the base bandage 10, as depicted in FIG. 6). In this manner, the user 20 may replace the top bandage 24 with the other top bandage 28 without causing any inconvenience or discomfort to the user 20. The base bandage 10 may remain stationary and the top bandage 24 may be replaced without replacing/removing the base bandage 10.

FIG. 7 depicts a top view of an adhesive base bandage 30 in accordance with the present disclosure. FIG. 7 will be described in conjunction with FIG. 8. The adhesive base bandage 30 may be similar to the adhesive base bandage 10. In some aspects, the adhesive base bandage 30 (or base bandage 30) may be circular in shape.

The base bandage 30 may include a top surface 32 and a bottom surface. The bottom surface may include an adhesive 34. The base bandage 30 may include an opening 36 that may be disposed at a center portion of the base bandage 30. The base bandage 30 may further include one or more tabs 38 that may be disposed at the top surface of the base bandage 30, which may facilitate sticking of the base bandage 30 to the user's skin, and also easy removal from the user's skin.

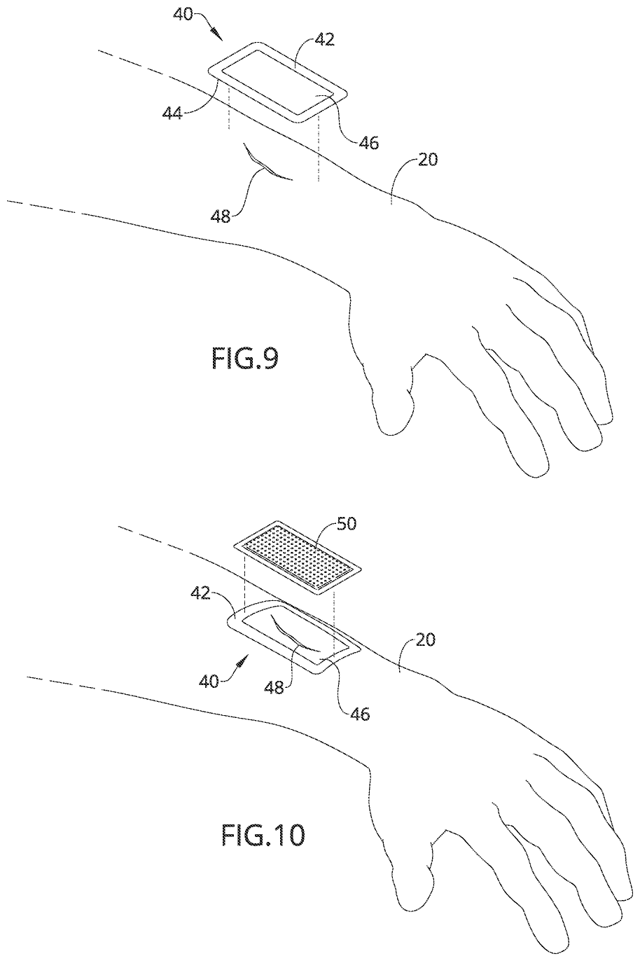

FIG. 9 depicts a top perspective view of an adhesive base bandage 40 applied on user's forearm in accordance with the present disclosure. FIG. 9 will be described in conjunction with FIG. 10. The adhesive base bandage 40 may be similar to the adhesive base bandage 10. In some aspects, the adhesive base bandage 40 (or base bandage 40) may be rectangular in shape.

The base bandage 40 may include a top surface 42 and a bottom surface. The bottom surface may include adhesive 44. The base bandage 40 may include an opening 46 that may be disposed at a center portion of the base bandage 40, which may expose a treatment area 48. The base bandage 40 may be applied to the user's skin via the adhesive 44. When the base bandage 40 may be applied, the user may apply a top bandage 50 over the base bandage 40 in the similar manner as described above and as depicted in FIG. 10.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

The claimed invention is:

1. A wound dressing comprising:
a base bandage having a top surface and a bottom surface, wherein the base bandage is configured to adhere to a user's skin via the bottom surface; and
a top bandage having a top surface and a bottom surface, wherein the bottom surface of the top bandage is configured to adhere to the top surface of the base bandage.

2. The wound dressing of claim 1, wherein the base bandage comprises an opening to expose a wound on the user's skin, and wherein the top bandage is configured to cover the wound.

3. The wound dressing of claim 2, wherein the opening is located at a center portion of the base bandage.

4. The wound dressing of claim 1, wherein the bottom surface of the base bandage comprises a first adhesive configured to stick the base bandage to the user's skin.

5. The wound dressing of claim 1, wherein the bottom surface of the top bandage comprises a second adhesive configured to stick the top bandage to the top surface of the base bandage.

6. The wound dressing of claim 1, wherein the base bandage comprises one or more tabs disposed at corners of the base bandage.

7. The wound dressing of claim 1, wherein a size of the base bandage is greater than a size of the top bandage.

8. A method to use a wound dressing, the method comprising:
applying a base bandage on a user's skin, wherein the base bandage comprises a top surface and a bottom surface, and wherein the base bandage is configured to adhere to the user's skin via the bottom surface; and
applying a first top bandage on the base bandage, wherein the first top bandage comprises a top surface and a bottom surface, and wherein the bottom surface of the first top bandage is configured to adhere to the top surface of the base bandage.

9. The method of claim 8 further comprising:
removing the first top bandage by detaching the first top bandage from the base bandage; and
applying a second top bandage on the base bandage.

10. The method of claim 8, wherein the base bandage comprises an opening to expose a wound on the user's skin, and wherein the first top bandage is configured to cover the wound.
